(19) United States
(12) Patent Application Publication
(10) Pub. No.: US 2022/0117859 A1
(43) Pub. Date: Apr. 21, 2022

(54) TOPICAL ANTIMICROBIAL MICROEMULSIONS WITH FLUORESCENT MATERIALS

(71) Applicant: 3M INNOVATIVE PROPERTIES COMPANY, St. Paul, MN (US)

(72) Inventors: Vinod P. Menon, Woodbury, MN (US); Ranjani V. Parthasarathy, Woodbury, MN (US); Maren L. David, Apple Valley, MN (US); Evan T. Roth, Mendota Heights, MN (US)

(21) Appl. No.: 17/298,057

(22) PCT Filed: Nov. 20, 2019
(86) PCT No.: PCT/IB2019/059988

§ 371 Date: May 28, 2021

Related U.S. Application Data
(60) Provisional application No. 62/774,028, filed on Nov. 30, 2018.
(60) Provisional application No. 62/774,768, filed on Dec. 3, 2018.

(51) Int. Cl. A61K 8/06; A61Q 17/00

(52) U.S. Cl. CPC A61K 8/068; A61K 2800/434; A61Q 17/005

(57) ABSTRACT

A topical antimicrobial composition includes a lipophilic component including a surfactant system with an HLB value of less than 10; an amphiphilic component including an antimicrobial compound; an aqueous hydrophilic component; and a fluorescent material. The composition includes less than 10 wt % of lower monohydric alcohols based on the total weight of the composition. The composition is in the form of a microemulsion at room temperature with a disperse phase having droplets with a particle size of 5 nm to 400 nm.

### BACKGROUND

Routine pre-operative cleansing of the skin at an operative site with an antiseptic is important to prepare a patient for a surgical procedure. The purpose of preoperative skin antisepsis is to reduce the bioburden of microorganisms on the skin and thus reduce the risk of inoculation of the surgical site with potentially infecting organisms that reside on the skin.

Some common preoperative skin preparations include lower monohydric alcohols such as, for example, isopropyl alcohol (IPA), in combination with antiseptic compounds such as chlorhexidine, and iodine/iodophors. These preoperative skin preparations are fast-acting antiseptics (due to the alcohol) with persistent activity (due to the chlorhexidine or iodophor). They are effective against gram positive and gram negative bacteria, fungi and most viruses. However, preoperative skin preparations including lower monohydric alcohols such as IPA can be flammable, which presents as a potential patient safety issue when adequate attention is not given to dry time requirements, or appropriate application technique to avoid pooling.

In some cases, patients can use aqueous antiseptic products to cleanse their body prior to surgery. Many chlorhexidine products are sold today in a bottle or impregnated in a wipe that are used multiple days prior to surgery to reduce the risk of surgical site infection. Since the antiseptic product is used throughout the body, neck down, and used multiple times a day in hospitals, it is necessary to make sure that the skin is not irritated and that the cleansing process leaves the patient feeling comfortable with a good bathing experience. Certain oils as emollients can help with good skin care, but are generally incompatible with aqueous solutions. In some cases, macroemulsions can be prepared but they generally suffer from stability issues (for example, separation of the components). Also, in the case of pre-impregnated wipes, a white milky fluid coming out of the wipe during application may not give the feeling of being bathed properly, especially if the fluid is a “leave-on” product that is intended to be left on the skin after application.

Some topical preoperative skin preparations are oil-in-water or water-in-oil emulsions, which form when a small amount of an appropriate surfactant is mechanically agitated with oil and water. The emulsions are two-phase dispersions in which one discontinuous phase exists as droplets coated by surfactant that is dispersed throughout a second continuous phase. Emulsions have a droplet size of about 0.1 micron to about 1 micron, and are typically milky or turbid in appearance.

If a suitable surfactant with an appropriate balance of hydrophilic and lipophilic properties is selected for use in the emulsion, and the selected surfactant is used in the right concentration, a microemulsion can be formed. In the microemulsion the surfactant generates an ultra-low free energy per unit of interfacial area between the oil and water phases that improves stability and only requires gentle mixing to form. Microemulsions have a smaller particle size than the milky emulsions, on the order of less than about 400 nm, and the small particle size makes the microemulsions appear translucent or even transparent to visible light.

In some cases there may be a need to determine if patients or other health care workers have complied with a bathing protocol using a skin cleansing emulsion. For example, it may be important to determine if epithelial surfaces have been wet completely over a desired region of the body of a patient. The problem of compliance may include patient bathing prior to surgery, or surgical site disinfection from the nurse that administers a skin disinfection protocol in the pre-op area of the hospital or the intensive care unit (ICU).

### SUMMARY

A visible dye, which has an absorption (and transmission) peak in the visible region of the electromagnetic spectrum (400-750 nm), may be included in an antimicrobial composition, or on the cloth, wipe, or mitt used for patient bathing, to determine whether the surgical site has been properly disinfected to reduce bacterial load on the skin. The presence of the dye on the skin would allow a health care professional to determine whether the patient had bathed properly prior to a medical procedure. However, a visible dye on the skin would require that the patient function with colored skin for at least one day, and perhaps for multiple days. In addition, some surgical prep solutions have an intense color that can give medical personnel a visual indication of whether the preps were applied correctly, and use of a visible dye in the patient bathing solution may also interfere with downstream activities such as prepping for a selected surgical procedure. Thus, for patient bathing and other medical cleansing applications, it may not be desirable to include a visible dye that leaves a pronounced stain on skin after application.

In one aspect, the present disclosure is directed to a non-flammable antimicrobial composition with fast acting, persistent, broad spectrum antiseptic activity that may be used as, for example, a preoperative skin preparation. The antimicrobial composition has excellent adhesion to surgical incise drapes, and dries quickly when applied to the skin of a patient. The antimicrobial composition includes a material that fluoresces in the visible region when exposed to ultraviolet radiation. The presence (or absence) of the antimicrobial composition on the skin of the patient can be monitored by medical personnel with an ultraviolet lamp. In some embodiments, the antimicrobial composition may optionally include a visible dye that fluoresces in the ultraviolet region, as long as the visible dye disappears on rubbing and does not leave a pronounced stain on skin.

The topical preoperative skin preparation is in the form of a microemulsion at room temperature, and has a disperse phase with droplets having a particle size of about 5 nm to about 400 nm. The microemulsion includes a surfactant system with an HLB value of less than 10, as well as an antimicrobial compound that in some embodiments can act as a co-surfactant to enhance the thermodynamic stability of the microemulsion. The microemulsion forms an antiseptic preoperative skin preparation product that is stable for up to about 2 years at room temperature.

The antimicrobial composition includes at least one of a visible or a fluorescent dye/pigment, which can be used to provide a visible indication of compliance with, for example, a pre-operative bathing or other medical skin cleansing procedure. The antimicrobial composition of the present disclosure is particularly useful in that both water soluble and oil soluble dyes can be introduced into their respective water phase or oil phases of the microemulsion. In some embodiments, even dyes or pigments that are generally not compatible with each other can be introduced into two separate phases that could then result in one stable microemulsion.

The intimate contact of hydrophilic and hydrophobic domains in the microemulsion results in a microscopically amphiphilic composition that provides excellent skin wetting. The microemulsion also has a low viscosity, which makes it possible to dispense the topical skin preparation product with a gravity-fed single dose sterile applicator, or to quickly and easily apply the product on the skin of a patient with a mitt, a sponge, or a cloth. The microemulsion forms spontaneously, which makes the topical skin preparation product relatively simple to manufacture at low cost.

In one aspect, the present disclosure is directed to a topical antimicrobial composition including a lipophilic component including a surfactant system with an HLB value of less than about 10; an amphiphilic component including an antimicrobial compound; an aqueous hydrophilic component; and a fluorescent material. The composition includes less than about 10 wt % of lower monohydric alcohols, based on the total weight of the composition. The composition is in the form of a microemulsion at room temperature with a disperse phase having droplets with a particle size of about 5 nm to about 400 nm.

In another aspect, the present disclosure is directed to a topical mammalian tissue antiseptic composition, the composition including: about 2 wt % to about 50 wt %, based on the total weight of the composition, of an ester chosen from isopropyl myristate, isopropyl palmitate, dibutyl adipate, diisobutyl adipate, monoalkyl glycols; glycerol alkyl ethers; monoacyl glycerols, and mixtures and combinations thereof, about 0.5 wt % to about 10 wt %, based on the total weight of the composition, of an antimicrobial compound chosen from biguanides, (bis)biguanides, polymeric biguanides, quaternary compounds, octenidine, and mixtures and combinations thereof, about 5 wt % to about 98 wt %, based on the total weight of the composition, of an aqueous hydrophilic component including at least 80 wt % water, based on the total weight of the aqueous hydrophilic component; and a fluorescent material. The composition includes less than about 5 wt % of lower monohydric alcohols, based on the total weight of the composition. The composition is in the form of an oil in water microemulsion at room temperature, and has a disperse phase having droplets with a particle size of about 5 nm to about 400 nm.

In another aspect, the present disclosure is directed to a method of disinfecting mammalian skin, the method including: applying to the mammalian skin a topical antiseptic composition including: a lipophilic component including a surfactant system with an HLB value of less than about 10; an amphiphilic component including an antimicrobial compound; an aqueous hydrophilic component; and a fluorescent dye. The composition includes less than about 10 wt % of lower monohydric alcohols, based on the total weight of the composition. The composition is in the form of a microemulsion at room temperature with a disperse phase having droplets with a particle size of about 5 nm to about 400 nm.

In another aspect, the present disclosure is directed to a kit of parts, including: a topical mammalian tissue antiseptic composition, including: a lipophilic component including a surfactant system with an HLB value of less than about 10; an amphiphilic component including an antimicrobial compound; an aqueous hydrophilic component; and a fluorescent dye. The composition includes less than about 10 wt % of lower monohydric alcohols, based on the total weight of the composition. The composition is in the form of a microemulsion at room temperature with a disperse phase having droplets with a particle size of about 5 nm to about 400 nm. The kit further includes an applicator for applying the antiseptic composition to skin of a patient, and an optional surgical incise drape.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a schematic diagram of components of a kit including applicators that can be used to apply the antimicrobial compositions of the present disclosure to the skin of a patient.

FIG. 2A is a photograph of the formulations of Example 1 immediately after mixing, while FIG. 2B is a photograph of the formulations of Example 1 on standing.

FIG. 3 is a pseudo-ternary phase diagram of aqueous CHG, CAPMUL MCM monoglyceride mix, and isopropyl monostearate from Example 2, with the tieline composition shown on the right.

FIG. 4 is a pseudo-ternary phase diagram of aqueous CHG, CAPMUL MCM NF monoglyceride mix, and isopropyl monostearate from Example 2, with the tieline composition shown on the right.

FIG. 5 is a pseudo-ternary phase diagram of aqueous CHG, CAPMUL MCM (C8) EP monoglyceride mix, and isopropyl monostearate from Example 2, with the tieline composition shown on the right.

FIG. 6 is a plot with curves showing the transition from macroemulsion (below the line) to microemulsion (above the line) as a function of CHG and monoglyceride concentration in the presence and absence of benzyl alcohol.

FIG. 7 is a plot comparing the adhesion of pig skin to a surgical incise drape with a CHLOROPREP control vs. the antimicrobial composition of Example 4.

FIG. 8 is a plot of ex vivo antimicrobial efficiency of the antimicrobial compositions of Example 5 vs. a CHLOROPREP control.

Like symbols in the drawings indicate like elements.

### DETAILED DESCRIPTION

In one aspect, the present disclosure is directed to topical antimicrobial compositions that can provide pre-surgical tissue antiseptic compositions, personal care compositions, transdermal drug delivery compositions, and the like. The topical antimicrobial compositions are in the form of a microemulsion at room temperature having a discontinuous or disperse phase with droplets having a particle size of about 5 nm to about 400 nm.

The antimicrobial composition includes a lipophilic component, a hydrophilic component, and an antimicrobial amphiphilic component, and in some embodiments forms spontaneously when the components are combined and mixed with each other, without requiring high energy input as is normally required for the formation of an emulsion having droplets with larger particle sizes. The antimicrobial composition may have a colloidal lipophilic phase dispersed in a hydrophilic phase, or a hydrophilic phase colloidally dispersed in a lipophilic phase. In various embodiments, the size of the dispersed phases is usually in the range from about 5 nm to about 400 nm, or about 5 nm to about 200 nm, or about 5 nm to about 100 nm, as measured with, for example, techniques such as Cryo-Transmission Electron Microscopy.

In terms of its rheological properties, the antimicrobial composition may be in the form of a liquid or a gel, i.e. in liquid or semisolid form.

The antimicrobial composition is optically isotropic, which is this application refers to a material having physical properties that are substantially the same when measured in different directions. In some embodiments, the antimicrobial compositions are translucent to visible light (about 400 nm to about 750 nm) at room temperature, which means that the microemulsions pass visible light but do not allow viewing of detailed images therethrough. In other embodiments, the antimicrobial compositions appear substantially transparent or clear to visible light at room temperature. In this application the term substantially transparent refers to materials that pass visible light in the wavelength region sensitive to the human eye, while rejecting light in other regions of the electromagnetic spectrum. In some embodiments, the reflective edge of the antimicrobial composition should be above about 750 nm, just out of the sensitivity of the human eye.

The antimicrobial compositions are stable, which in the present application means that the antimicrobial composition remains optically isotropic and in its as-formulated translucent or substantially transparent form for a period of at least about 6 months at room temperature (±1 month). In some embodiments, the antimicrobial composition is stable for a period of about 6 months to about 2 years at room temperature (±1 month). In this application, stable means that the antimicrobial composition remains a microemulsion and does not separate into discrete oil and water phases upon standing at room temperature.

The antimicrobial composition includes a lipophilic component including a surfactant system with an HLB value of less than about 10, an amphiphilic component including an antimicrobial compound; and an aqueous hydrophilic component. The antimicrobial composition includes less than about 10 wt % of lower monohydric alcohols, based on the total weight of the composition, and as such has low flammability.

In various embodiments, the aqueous hydrophilic component is present in the antimicrobial composition in an amount of about 5 wt % to about 98 wt %, or about 10 wt % to about 90 wt %, based on the total weight of the composition (±5%). In various embodiments, the aqueous hydrophilic component includes at least about 80 wt % water, or at least about 90% water, based on the total weight of the aqueous hydrophilic component (±5%). In some embodiments, the aqueous hydrophilic component consists of water, which in this application means that the aqueous hydrophilic component is substantially 100% water, or 100% water, based on the total weight of the aqueous hydrophilic component (±1%). In some embodiments, the aqueous hydrophilic component includes predominantly aqueous solutions such as buffers.

In some embodiments, the aqueous hydrophilic component in the antimicrobial composition further includes a humectant. As used herein the term “humectant” refers to polar compounds or mixtures of compounds that act to retain or absorb moisture. Suitable humectants include, but are not limited to, polyols, such as glycerin, propylene glycol, dipropylene glycol, polypropylene glycol, glycerine ethoxylates, methyl glucose ethoxylates, polyethylene glycol, polyethylene/polypropylene glycols, and sorbitol. In some embodiments, the humectants include liquid polar solvents such as for example, monoalkyl glycols, glycerol alkyl ethers, monoacyl glycerols, and mixtures and mixtures and combinations thereof. Suitable examples of the liquid polar solvents include, but are not limited to, glycerol, propylene glycol, polyethylene glycol, pentylene glycol, and mixtures and combinations thereof.

Diols such as propylene glycol and pentylene glycol are well tolerated by the skin, and have high affinity to skin and hair. In some embodiments, the diols have a small relatively lipophilic molecular region by virtue of which they may also be considered as somewhat amphiphilic, thus enforcing the functionality of the amphiphilic component of the composition and enhance the solubilization of poorly water-soluble ingredients. In some embodiments, the diols can have substantial antimicrobial properties so that they allow for the formulation of aqueous topical compositions without any further preservatives, or with reduced preservative levels.

In some embodiments, the hydrophilic component is a mixture of water and a liquid glycol such as, for example, propylene glycol, pentylene glycol and mixtures thereof. For such mixtures, the ratio of water to glycol (or glycols) may be about 1:10 to about 10:1, or about 1:8 to about 8:1, or about 1:5 to about 5:1. Examples of useful hydrophilic components include water and pentylene glycol (2:1), water and propylene glycol (1:2) In various embodiments, the liquid glycol is present in the antimicrobial composition in any amount of about 0 wt % to about 50 wt %, or about 1 wt % to about 30 wt %, or about 5 wt % to about 20 wt % (±1%), based on the total weight of the antimicrobial composition. In some cases, aliphatic acids such as lactic acid, maleic acid, citric acid and aromatic organic acids such as salicylic acid can be added to the hydrophilic component to provide an enhancement in efficacy but typically at 5 wt % or lower of the formulation.

The addition of low levels of stabilizing ingredients in the hydrophilic component water phase can also be advantageous. Salts such as magnesium sulfate may be useful microemulsion stabilizers, and they do not significantly affect the water resistance of the formulations. However, the addition of magnesium sulfate can, in some instances, inactivate bioactive agents, e.g., antimicrobial agents such as chlorhexidine gluconate (CHG). The addition of water-soluble gums such as guar derivatives, xanthan gum, and thickeners such as hydroxy ethyl cellulose, hydroxy propyl cellulose and carboxyl vinyl polymers may be helpful in stabilizing the microemulsion. Suitable oil phase emulsion stabilizers include, but are not limited to, ethylene/acrylic acid copolymers such as those available under the trade designation AC540 from Allied Signal, Morrison, N.J., and N-vinyl pyrrolidone/olefin copolymers such as that available under the trade designation GANEX V-216 from ISP International Specialty Products, Wayne, N.J. In some cases, addition of non-ionic surfactants like tween or pluronic can be used to help prepare the microemulsion.

The antimicrobial composition further includes a lipophilic component, which substantially contributes to the formation of a colloidally dispersed lipophilic phase in the microemulsion. In some embodiments, the lipophilic component is selected to yield a dispersed lipophilic phase in the microemulsion, so that the composition is in the form of an oil-in-water microemulsion (o/w-microemulsion).

The lipophilic component includes a surfactant system with an HLB value of less than about 10. The surfactant system can include one or more surfactants. It is noted that some of the commonly used surfactants in the pharmaceutical or cosmetic field are in fact mixtures of chemically related molecules. It is also noted that the technical literature relating to microemulsions often refers to surfactants and co-surfactants, even in the absence of functional differences between them, whereas in the present application surfactants are simply termed as such, without using the term co-surfactant. Surfactants may also be referred to herein as emulsifiers.

The surfactant system can include an excipient suitable for pharmaceutical use, and should include surfactants that are physiologically well tolerated after administration to the skin and/or a mucosa.

The surfactant system includes at least one surfactant with an HLB value of less than about 10, or less than about 9, or less than about 7, or less than about 6, or less than about 1.5, or less than about 1. The HLB value (Hydrophile-Lipophile Balance) is an empirical expression for the relationship of the hydrophilic and hydrophobic groups of a surfactant, and in most cases the higher the HLB value, the more water-soluble the surfactant. The HLB system is particularly useful to identify surfactants for oil and water emulsification. While the antimicrobial composition may be a water-in-oil microemulsion in which water is dispersed in oil, or an oil-in-water microemulsion in which oil is dispersed in an aqueous phase, in most cases oil-in-water microemulsions are preferred. Suitable HLB values may vary depending on the oil phase components, e.g., more polar oils may require higher HLB polymers. Also, the selected range of HLB values may vary depending on other additives, which may optionally be added to the emulsion formulation.

In the present disclosure, HLB values are calculated using the method of Griffin (Griffin W C; J. Soc. of Cosmetic Chemists, pp. 249-256 (1954)). Thus, as used herein, the “HLB Method” involves a calculation based on the following: HLB=(E+P)/5, where E is the weight percent of oxyethylene content and P is the weight percent of polyhydric alcohol content (glycerol, sorbitol, etc.). For the compounds herein, glycerol segments with two hydroxyl groups, glycerol segments with one hydroxyl group, and hydroxyl-containing segments of any additional polyhydric molecules were included in the definition of P.

Other methods of calculating HLB are available and may be required when determining the HLB value for compounds lacking both E and P groups, as defined above. While the calculated value of HLB may vary depending on the method used, the trends and relative hydrophobicity of materials are expected to be similar.

The lipophilic component may include any suitable lipophilic compound or mixture of compounds capable of forming the lipophilic phase. The compounds making up the lipophilic component can be selected from a wide variety of oils or mixtures of oils that are conventionally used in the cosmetic art. Suitable oils include “emollient oils,” which as used herein refers to any dermally acceptable oil or mixture of oils which forms a barrier on the skin capable of retarding the evaporation of water from the skin. The oil base of the microemulsions can be solid or liquid, but the entire antimicrobial composition should be somewhat fluid at skin temperatures for ease of application.

Examples of suitable oils for the lipophilic component include silicone fluids, saturated fatty esters and diesters such as diisopropyl adipate, dicapryl adipate, diisopropyl sebacate, dioctyl sebacate, dioctyl ether, glyceryl tricaprylate/caprate, diethyleneglycol dicaprylate/caprate, propylene glycol dipelargonate, polyalkoxylated alcohols such as 15 mole propoxylate of stearyl alcohol, paraffin oils and waxes, animal and vegetable oils including mink oil, coconut oil and derivatives thereof, palm oil, corn oil, cocoa butter, petrolatum, coconut oil, sesame oil, and the like, lanolin derivatives, fatty alcohols such as isostearyl alcohol, isocetyl alcohol, cetyl/stearyl alcohol, and straight chain alcohols from C6-C18 and certain petroleum distillates which are toxicologically safe such as C8-C22 isoparaffin hydrocarbon solvents, e.g., isooctane and isododecane, mixtures of mono, di and tri glycerides of long chain fatty acids, mixtures of propylene glycol mono, di and tri esters of fatty acids. In some embodiments, the same excipients can acts as the amphiphilic component of the composition, depending on how they are formulated (i.e. the remaining excipients).

Suitable lipophilic components include compounds that are well tolerated by the skin and/or mucosae, and include, but are not limited to, esters, ethers, glycols, amides, monoalkyl and monoalkylene alcohols with greater than 7 carbon atoms and less than 18 carbon atoms, liquid paraffins, liquid waxes, and mixtures and combinations thereof. In some embodiments, which are not intended to be limiting, the lipophilic component includes esters chosen from isopropyl myristate, isopropyl palmitate, dibutyl adipate, diisobutyl adipate, methyl behenate, methyl stearate, arachidyl propionate, behenyl lactate, stearyl acetate, 2 mole propoxylate of myristyl propionate, cetyl palmitate, butyl stearate, and glycerol monoerucate. The oils mentioned in this list are merely examples and are not intended to be limiting. Examples of suitable ethers include, but are not limited to, ethylhexyl glycerin. Examples of suitable glycols include, but are not limited to, 1,2 octane diol, 1,2 decane diol, and mixtures and combinations thereof

In some embodiments, the lipophilic component includes esters of glycerol with a fatty acid. In various embodiments, the fatty acid is chosen from oleic, linoleic, linolenic, caproic, caprylic, capric, lauric, and mixtures and combinations thereof. In some embodiments, the fatty acid is chosen from caprylic, capric, and mixtures and combinations thereof. In various embodiments, the fatty acid is present at about 0.5 wt % to about 15 wt %, or about 1 wt % to about 10 wt %, based on the total weight of the antimicrobial composition.

In some embodiments, the surfactant system includes a mixture of monoglycerides, diglycerides, and triglycerides of caprylic acid and capric acid. For example, the surfactant can include at least 80% by weight of a mixture of monoglycerides of caprylic acid, and up to about 20% by weight of a mixture of monoglycerides of capric acid. In another example, the surfactant can include at least 90% by weight of a mixture of monoacylglycerols, diacylglycerols, and triacylglycerols of caprylic acid, and about 10% by weight of a mixture of monoacylglycerols, diacylglycerols, and triacylglycerols of capric acid. In yet another example, the surfactant can include at least about 95% by weight of a mixture of monoacylglycerols, diacylglycerols, and triacylglycerols of capric acid, and about 5% by weight of a mixture of monoacylglycerols, diacylglycerols, and triacylglycerols of caprylic acid.

In some embodiments, the surfactant system includes an optional surfactant chosen from alcohols such as, for example, benzyl alcohol, phenoxy ethanol, and combinations thereof. In one non-limiting example, the alcohol is present in the composition at about 1 wt % to about 5 wt %, based on the total weight of the composition.

The lipophilic component is present in the antimicrobial composition at about 2 wt % to about 50 wt %, about 5 wt % to about 35 wt %, or about 2 wt % to about 30 wt %, based on the total weight of the composition (±1%). In various embodiments, the content of the lipophilic component is kept at or below about 50 wt % relative to the total composition to allow for an increased amount of the hydrophilic component.

In a further aspect, in some embodiments the antimicrobial composition includes a larger amount of hydrophilic component than of lipophilic component, i.e. the ratio of hydrophilic to lipophilic component is 1:1 or higher, such as, for example, in the range from about 1:1 to 3:1.

In some embodiments, the addition of a silicone oil such as dimethicone to the lipophilic component to prepare the microemulsion can also be advantageous in improving the ability of the antimicrobial compositions to act as a barrier to urine, feces, or other indigenous and exogenous materials when used as moisturizing compositions (e.g., moisturizing skin treatments). In some embodiments, the dimethicone may be present at about 1 wt % to about 5 wt %, based on the total weight of the composition (±1%). In some embodiments, aloe may be used to help improve the solubility of the dimethicone in the composition to provide further moisturization.

In some embodiments, the lipophilic component may include auxiliary emulsifiers conventionally used in cosmetic formulations to ensure stability and extend shelf life of any of the compositions of the present invention. Suitable auxiliary emulsifiers include, but are not limited to, C12-C18 alkyl carboxylic acids such as stearic acid, polypropylene glycol (PPG) (15) stearyl ether (commercially available under the trade designation ARLAMOL E from Uniqema, Wilmington, Del.), and 20-mole ethoxylate of cetyl/stearyl alcohol, polyetherpolyester polymer, such as polyethylene glycol (PEG) (30) polyhydroxy-stearate, MW of approximately 5000 (commercially available under the trade designation ARLACEL P135 from ICI, Wilmington, Del.). In various embodiments, the auxiliary emulsifier is preferably present in an amount of about 1 wt % to about 20 wt %, or about 5 wt % to about 10 wt %, based on the total weight of the antimicrobial composition.

The amphiphilic component of the antimicrobial composition includes at least one antimicrobial compound. In some embodiments, the antimicrobial compound includes iodine and its complexed forms, which are commonly referred to as iodophors. Iodophors are iodine complexes with polyethylene glycol and its derivatives, N-vinyl caprolactam containing polymers such as polyvinylpyrrolidone, as well as other polymers that tend to hydrogen bond with hydrogen iodide or hydrogen triiodide or complex with salts such as sodium or potassium triiodide. In some embodiments, the iodophor is povidone-iodine, and most preferably povidone-iodine USP.

Other suitable antimicrobial compounds include chlorhexidine salts; octenidine salts, parachlorometaxylenol (PCMX); triclosan; hexachlorophene; fatty acid monoesters of glycerin and propylene glycol such as glycerol monolaurate, glycerol monocaprylate, glycerol monocaprate, propylene glycol monolaurate, propylene glycol monocaprylate, propylene glycol monocaprate; phenols; surfactants and polymers that include a C12-C22 hydrophobe and a quaternary ammonium group; polyquaternary amines such as polyhexamethylene biguanide; quaternary silanes; hydrogen peroxide; silver and silver salts such as silver chloride, silver oxide and silver sulfadiazine; and the like.

In some embodiments the antimicrobial compound is chosen from biguanides, (bis)biguanides, and mixtures and combinations thereof. In some embodiments, the biguanides and (bis)biguanides may include polymeric biguanides, polymeric (bis)biguanides, and mixtures and combinations thereof. In some embodiments, the antimicrobial compound is chosen from polyhexamethylene biguanide (PHMB), chlorhexidine, octenidine, quaternary compounds such as benzalkonium chloride, and mixtures and combinations thereof. In some embodiments, the chlorhexidine is a soluble salt, and the diacetate and digluconate salts have been found to be particularly useful in the antimicrobial composition. In various embodiments, octenidine could be in the form of the dihydrochloride or other suitable salts that can improve solubility in the microemulsion. In some embodiments, the antimicrobial compound includes chlorhexidine gluconate (CHG), also referred to as chlorhexidine digluconate, or consists of CHG. CHG is a chemical antiseptic that is effective on both gram-positive and gram-negative bacteria. CHG is both bacteriocidal (kills) and bacteriostatic (stops reproductions) of any bacteria on mammalian skin.

In some embodiments, which are not intended to be limiting, the antimicrobial compound is present in the antimicrobial composition at about 0.05 wt % to about 10 wt %, or about 0.1% wt % to about 5 wt %, or about 1 wt % to about 3 wt %, or about 1.5 wt % to about 2.5 wt %, based on the total weight of the composition (±1%).

The antimicrobial composition further includes less than about 10 wt %, or less than about 5 wt %, or less than about 1 wt %, or about 0 wt %, of lower monohydric alcohols, based on the total weight of the composition (±1%). In the present application the term lower monohydric alcohols refers to alcohols with a single hydroxyl group and the formula CₙH₂ₙ₊₁OH, wherein n=2 to 5, such as, for example, methanol, ethanol, propanol, isopropyl alcohol, and the like. For example, in some embodiments the antimicrobial composition includes up to about 5 wt %, or up to about 4 wt %, or up to about 3 wt %, of a lower monohydric alcohol such as, for example, isopropanol, which can provide the composition with properties such as enhanced mold resistance.

The reduced amount of C2-C5 monohydric alcohols provides the antimicrobial composition with good flammability properties when used in a medical or surgical setting, particularly when electrocautery procedures are performed. In some embodiments, for example, the antimicrobial composition has no closed cup flash point at temperatures of 70° F. to 200° F. as measured according to ASTM D-3278-96 e-1.

In various embodiments, the antimicrobial compositions may include further ingredients as required. For example, the antimicrobial compositions may optionally include a further active ingredient, e.g. a corticosteroid, an antibiotic, an antimycotic, and/or an antiviral agent.

The antimicrobial composition may further include up to about 5 wt %, or up to about 4 wt %, or up to about 3 wt %, based on the total weight of the composition, of other optional ingredients including, for example, agents for adjusting the pH (e.g. acids, buffer salts, bases), antioxidants (e.g. ascorbic acid, vitamin E and its derivatives, BHT, BHA, disodium EDTA, etc.), preservatives (e.g. cationic surfactants such as benzalkonium chloride; benzyl alcohol, sorbic acid etc.), permeation enhancers (DMSO, diethylene glycol monoethyl ether (DEGEE) available under the trade designation TRANSCUTOL from Gattefossé, Paramus, N.J., menthol, oleic acid, n-alkanols, dimethyl isosorbides, 1-alkyl-2-pyrrolidones, N,N-dimethlyalkanamides, and 1,2-alkanediols, etc.), and the like.

Other materials conventionally used in cosmetic compositions such as waxes, film-forming polymers, propellants, buffers, organic or inorganic suspending or thickening agents, plasticizers, and herbal extracts can also be included in minor amounts in the antimicrobial compositions, preferably in amounts that do not adversely affect the substantivity of the compositions. These materials can be added to the aqueous or oil phase (depending on solubility) prior to emulsification, or added after the emulsions have been prepared and cooled. The latter is preferred when materials with heat sensitivity are used.

In some embodiments, the antimicrobial compositions may be applied directly on mammalian skin, mucosal tissue or hair to disinfect the site. In various embodiments, the antimicrobial compositions may be applied using a wide variety of applicators including, but not limited to, foam applicators, sponges, a woven or nonwoven cloth, a woven or non-woven mitt, and the like. In some embodiments, the foam applicator includes a compressed foam. In various embodiments, the compressed foam is at least a 2× compressed foam, or is compressed at about 2× to about 6×. In some embodiments, the antimicrobial composition may be supplied as a layer on a surface of a surgical incise drape or a surgical tape, or may be impregnated into the surface of the surgical incise drape or a surgical tape.

In various embodiments, the amphiphilic component and the antimicrobial compound are present in the antimicrobial composition in an amount sufficient such that the antimicrobial composition provides at least a 1.5-log microbial reduction on mammalian skin following 10 minute contact as measured according to ASTM E1874-09. In some embodiments, the composition provides at least a 2-log microbial reduction on mammalian skin following 10 minute contact as measured according to ASTM E1874-09.

The antimicrobial composition is also highly persistent on the surface of mammalian skin, skin mucosae, or hair. In this application persistence refers to microbial counts not returning to baseline at a set time, for example 24 hour persistence would be that for 24 hours, the microbial counts has not returned to what it was prior to treatment. Efficacy for 24 hours refers to having low bacterial bioburden for a period of 24 hours. A formulation that has high efficacy at 24 hours means that it has very few bacteria left on skin after a period of 24 hours.

In some embodiments, the antimicrobial composition prevents microbial counts from returning to baseline for at least 24 hours, at least 48 hours, or at least 72 hours. In some embodiments, the antimicrobial composition has excellent efficacy for a period of at least 24 hours, at least 48 hours, or at least 72 hours. In some embodiments, the antimicrobial composition has both persistence and high efficacy for a period of at least 24 hours, at least 48 hours, or at least 72 hours.

When applied to mammalian (preferably, human) skin (or other tissue such as mucosal tissue or hair), the antimicrobial compositions form an oil film on the tissue surface. In spite of the oiliness and moisturizing effects of the microemulsions, pressure sensitive adhesives, such as used on medical tapes, IV site dressings, and surgical incise drapes, adhere at least as well and, in most cases, more strongly, to the emulsion-treated tissue (typically, skin) than to untreated tissue (typically, skin). Medical tapes and dressings that adhere particularly well to the microemulsions include those utilizing acrylate, block copolymer (e.g., adhesives based on KRATON polymers commercially available from Kraton Polymers, Houston, Tex.) and rubber based pressure sensitive adhesives. Suitable examples include, but are not limited to, tapes and dressings commercially available from 3M Company under the trade designations TRANSPORE, BLENDERM, STERI-STRIPS, MICROPORE, TEGADERM, STERIDRAPE, and IOBAN II.

A pressure sensitive adhesive article (e.g., tape, incise drape, wound dressing, and the like) applied over the antimicrobial compositions on mammalian tissue, typically skin (after allowing the emulsion or composition containing the emulsion to dry for at least 15 seconds), preferably adhere at a level of at least about 50% of the level of adhesion of the pressure sensitive adhesive article applied directly to the tissue, typically skin (i.e., without the emulsion).

For example, the level of adhesion provided by the compositions can be measured by applying a thin uniform amount to skin, applying the adhesive article, and rolling with a 4.5-pound (2.1-kg) 2-inch (5.1-cm) wide roller. After waiting 1-5 minutes the adhesive article is removed at a peel angle of 90° to the skin at a pull rate of 1 inch per minute according to a modified test procedure from J. Bone, Joint Surg. Am. 2012 Jul. 3; 94(13): 1187-92. In various embodiments, the antimicrobial composition adheres a surgical drape to mammalian skin at greater than about 80 grams per 0.5 inches, or 65 grams per cm.

The antimicrobial compositions, if applied in a thin film to mammalian tissue, typically skin, allow instantaneous adhesion of medical adhesive products. For example, within about 60 seconds, and often, in as little as 15 seconds, of application of a thin film, an adhesive product can be applied over the antimicrobial composition that will exhibit good adhesion in as little as about 5 minutes, or as little as about 60 seconds, or as little as about 40 seconds. In many of the preferred cases the adhesion over the antimicrobial compositions will exceed that of the product applied to dry unprepared tissue (typically skin).

The oil phase used in the water-in-oil emulsions of the present invention are preferably compatible with the medical pressure sensitive adhesives that may be placed over the composition. Not all oils will be compatible (i.e., allow good adhesion of the article) with all adhesives. For polyacrylate-based pressure sensitive adhesives, the oil phase preferably contains an ester-functional emollient oil or other emollient oil that is capable of plasticizing the adhesive, such as those described in U.S. Pat. No. 5,951,993 (Scholz et al.). For example, with most pressure sensitive adhesives that include predominantly alkyl acrylates, such as isooctylacrylate or 2-ethylhexylacrylate, emollient oils such as glyceryl tricaprylate/caprate, diiospropylsebacate, isopropylplamitate, diisopropyl adipate, diethyleneglycoldioctanoate/diiosnonanoate, and the like, are very effective. In some embodiments, ether-based emollient oils can be used. For example, with most polyacrylate pressure sensitive adhesives that include predominantly isooctylacrylate or 2-ethylhexylacrylate, dimethylisosorbide and PPG2 methyl ether are effective. Preferably, the ether-based emollient oil is not too polar. For example, materials such as glycereth 7 diisononanoate and glycerol triacetate may tend to reduce the adhesion of the medical pressure sensitive adhesive. It should be noted, however, that minor amounts of more polar components may be added to the oil phase and still allow good drape adhesion.

In some embodiments, if the continuous phase of the microemulsion is a water-insoluble oil, the adhesion of a medical adhesive product is not easily undercut by water or body fluids. This can be important for use of the antimicrobial compositions as a presurgical tissue antiseptic (“prep”), for use on skin or mucosal tissue (preferably, skin), over which an incise drape is optionally applied. In these surgical applications blood, saline, and other body fluids are constantly present which may tend to wash water-soluble preps away and perhaps even into the wound. The water-in-oil emulsion preps of the present invention, however, resist wash-off very well.

Furthermore, water resistance can also be important for preps over which an adhesive product is applied. For example, when using a surgical incise drape (adhesive coated film through which a surgical incision is made) adhesion to the antimicrobial composition throughout the surgery is important. Therefore, resistance to water and body fluid infiltration from the wound edge is important. This is similarly important for use around percutaneous devices such as a catheter insertion site, which can have fluid build-up around the catheter, which can affect adhesion. The adhesion of dressings such as thin film adhesive coated dressings over the antimicrobial compositions ensures a strong bond despite the presence of moisture.

Another advantage of some embodiments of the antimicrobial compositions, which can be particularly important for tissue antiseptics such as preoperative surgical preps and IV site preps, is that the emulsions may be removed gently with a cloth, gauze or other fabric optionally using a mild detergent for complete removal. No organic solvent-based removers are necessary but may be used if desired.

In some embodiments, the microemulsions may be used to form milks (i.e., low viscosity emulsions similar in consistency to cow's milk), lotions, and creams that are preferably water-repellent, moisturizing, and long lasting compared to most other commercially available skin lotions. These features are important for ostomy or incontinence applications where protection of the skin from irritating body fluids such as urine, feces, and intestinal fluids is desired. The fact that the microemulsions may enhance adhesion of pressure sensitive adhesives, allows them to be used to protect skin surrounding stomas, dermal ulcers, diseased skin, or surgical wounds without interfering with the application of adhesive wound dressings. This can also be an advantage over other percutaneous dressings when the present-invention emulsions are used in challenging fluid environments associated with surgical incise drapes, IV site dressings, and other dressings.

The antimicrobial composition includes at least one fluorescent material. In one embodiment, the fluorescent materials include at least one fluorescent compound such as, for example, a fluorescent dye, having an absorption peak in the ultraviolet and violet portion of the electromagnetic spectrum (340-370 nm) with a corresponding emission peak in the visible portion of the spectrum (400-750 nm), particularly in the blue region of the visible spectrum (420-470 nm), and can be monitored with an ultraviolet (UV) lamp. In another embodiment, suitable fluorescent materials include a fluorescent compound such as, for example, a fluorescent dye, having an emission peak in the near infrared (IR) region of the spectrum (780 nm to 2500 nm), which can be observed and detected with a suitable IR camera.

In some embodiments, the antimicrobial composition includes both a visible dye and a florescent dye. In some cases, the dye could be visible and fluorescent, and some amount of the dye could stain the wipe with rubbing and some be left on skin for observation with an ultraviolet lamp. In some embodiments, the fluorescent dye is also a visible dye, and the visible dye disappears on rubbing and does not leave a pronounced stain on the skin of a patient. In yet another embodiment, a visible dye can be used in combination with a fluorescent dye, and the latter may be rendered colorless after rubbing or after a certain period of time, while still maintaining an emission detectable by a UV lamp.

The dyes selected for incorporation into the antimicrobial composition should not inactivate the antimicrobial compounds therein. In addition, the dye that is delivered to skin should not substantially interfere with the visible dyes that are used for surgical preps. In various embodiments, the antimicrobial composition can include water soluble dyes, water insoluble dyes, and mixtures and combinations thereof. It is of course understood that the dye needs to be toxicologically safe and suitable for a drug product.

Suitable fluorescent dyes include, but are not limited to, stilbene compounds such as, for example, diaminostilbene disulphonic acid, coumarin derivatives, such as, for example, 4-methyl-7-diethylaminocoumarin, 1,3 diaryldipyrazoline derivatives, such as, for example, 1,3 diphenyl-4-methyl-5-alkylpyraZoline, naphthalimide derivatives, such as, for example, N-methyl-4 methoxynaphthalimide, and benzoxazole derivatives, such as, for example, 1,2-bis(5-methylbenZoxaZol-2-yl)ethylene. Pyrazoline derivatives (e.g. those available under the trade designation HOSTALUX PN, from Brenntag Great Lakes, LLC, Wauwatosa, Wis.), cationic benzimidazole derivatives (e.g. those available under the trade designation BLANKOPHOR ACR, from Tanatex Chemicals, Dalton, Ga.), hexasodium-2,2′-[vinylenebis[3-sulfonato-4,1-phenylene)imino[6-(diethylamino)-1,3,5-triazine-4,2-diyl]imino]]bis(benzene-1,4-disulphonate) compounds available under the trade designation TINOPAL SFP from BASF Ludwigshafen, Germany, and those available under the trade designation ULTRAPHOR ACR from Tanatex Chemicals. In some cases, curcuminoids including curcumin can be used as the fluorescent agent.

A wide variety of such compounds are available commercially from, for example, Keystone Aniline Corp. (Chicago, Ill.) Ciba Specialty Chemicals, (High Point, N.C.) and Sumita Optical Glass, Inc. (Saitama, Japan).

In one embodiment, the fluorescent compound, which can also be referred to as an optical brightener, can be a fluorescent glass. For example, in one embodiment the fluorescent glass includes fluorescent compounds that produce a green to bluish green fluorescence, and suitable examples include, a rare earth fluorescent glass, such as Lumilass G9 (Sumita Optical Glass, Saitama, Japan). In another example, the fluorescent glass may include an inorganic fluorescent glass, Lumilass B (Sumita), or red or orange fluorescent glasses such as those available, for example by Lumilass R7 from Sumita.

In another embodiment, the fluorescent material emits a blue fluorescence; examples of such compounds include, but are not limited to, a distearyl biphenyl derivative known as Tinopal CBS-X (Ciba), and an oxazole known as Keyfuor White.

The amount of the fluorescent compound in the antimicrobial composition may be varied depending upon the intensity of the fluorescence desired, and can be from about 0.0001 wt % to about 50 wt %; more typically, however, the amount used will be between about 0.001 wt % to about 10 wt %, or about 0.01 wt % to about 8 wt %, or about 0.05 wt % to about 5 wt %, based on the total weight of the composition (±1%).

The fluorescent compound can also be combined with other pigments or dyes, assuming the pigments and dyes are compatible with the antimicrobial compound. The additional color components can be either organic or inorganic. Examples of useful inorganic pigments include, but are not limited to, iron oxides (yellow, red, brown or black), ferric ammonium ferrocyanide (blue), manganese violet, ultramarine blue, chrome oxide (green), talc, lecithin modified talc, Zeolite, kaolin, lecithin modified kaolin, titanium dioxide (White) and mixtures thereof. Other useful pigments are pearlants such as mica, bismuth oxychloride and treated micas, such as titanated micas and lecithin modified micas. The organic pigments include natural colorants and synthetic monomeric and polymeric colorants. Exemplary are phthalocyanine blue and green pigment, diarylide yellow and orange pigments, and azo-type red and yellow pigments such as toluidine red, litho red, naphthol red and brown pigments. Also useful are lakes, which are pigments formed by the precipitation and absorption of organic dyes on an insoluble base, such as alumina, barium, or calcium hydrates. Polymeric colorants include nylon powder, polyethylene, and polyesters. With colorants, an exemplary list of cosmetically acceptable colorants can be found in the International Cosmetic Ingredient Dictionary and Handbook, 7th Edition, CTFA, 1997, pp. 1628-1630.

In various embodiments, the colorants other than the fluorescent brightener will normally constitute from about 0.1% wt % to about 30% wt % of the composition, the amounts varying depending upon the color desired.

Very few dyes/pigments are approved for drug use. Examples of such dyes include, but are not limited to, D&C green No. 8, FD&C Fluorescent Dye (DNC) or a Synthetic Organic Colorant 0.005-5.0% (MX659 Pylam-Cert Flourescent) (Day-Glo DG-00, A-594-5).

In some embodiments, the antimicrobial composition can incorporate a fluorescent dye, and can be delivered to the surface of the skin of a patient with a woven or non-woven cloth, a sponge, or a mitt to provide feedback during the rubbing process. The fluorescent dye has a faint color when initially applied to the skin, but disappears into skin by virtue of its composition and can later be probed with a UV lamp.

In one embodiment, the antimicrobial composition includes a fluorescent dye and a visible dye, and is provided in combination with a non-woven wipe or mitt. In various embodiments, at least greater than 70%, or greater than 90%, of the fluorescent dye is released from the wipe during the rubbing process, while the visible dye is reduced by at least 50%, or at least 90%, during the rubbing process to provide feedback on the efficacy of the bathing or skin surgical site disinfection protocol.

The dyes can be incorporated into the non-woven wipe or mitt in a wide variety of ways. For example, in some embodiments the dye can be encapsulated in a particle, which then fractures to release the dye during the skin rubbing procedure. In some embodiments, the dye can be closely associated with the antimicrobial compound (for example, CHG), so the dye location is indicative of the use of the antiseptic.

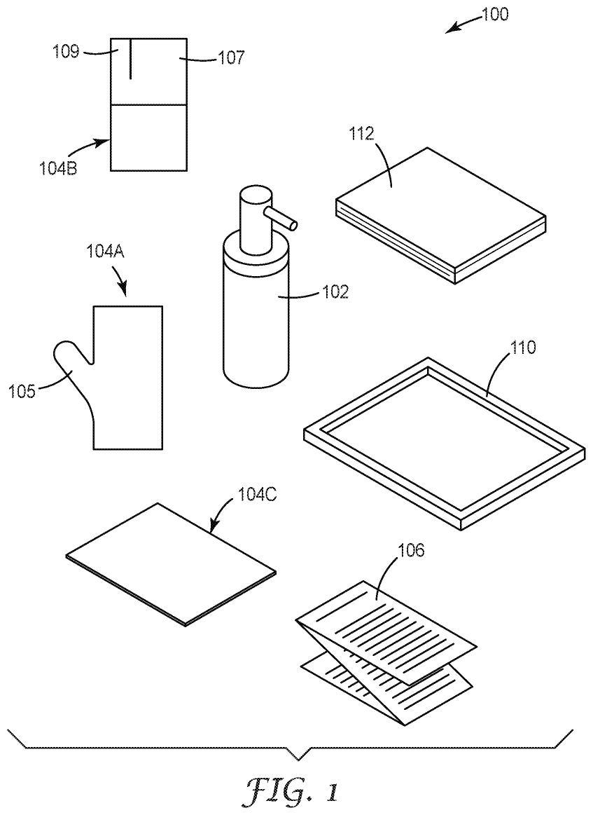

Referring to FIG. 1, in some example embodiments, the antimicrobial composition may be supplied in the form of a kit 100 including a container 102 of the antimicrobial composition and an applicator 104 that can be used to apply the antimicrobial composition to the skin. In some example embodiments, the container 102 may be a squeezable bottle or a collapsible tube, along with instructions 106 for proper application to the skin or to the included applicator 104. In one example, the applicator is a mitt such as shown in FIG. 1 as 104A, 104B. In another embodiment, the applicator may be a substantially flat cloth, wipe or sponge 104C. In another embodiment, the antimicrobial composition may be impregnated in a surface of the applicators 104A, 104B, 104C.

In some embodiments, the kit may be supplied in sterile form in a tray 110, and may optionally include the application instructions 106 along with a surgical incise drape 112. In some embodiments, the tray 110 and drape 112 may be packaged for a selected medical or surgical procedure.

The antimicrobial composition can be easily manufactured and scaled up into industrial scale production. The antimicrobial composition can be formed as the ingredients are combined and mixed together, even in the absence of high shear conditions or pressure homogenization. Therefore, the antimicrobial composition may be prepared using any standard mixing equipment which is suitable for the preparation of liquid pharmaceutical formulations at the appropriate scale. Optionally, ultrasound treatment of the combined ingredients may be used to accelerate the formation of a homogeneous microemulsion.

The water-in-oil microemulsions can be prepared by conventional methods, such as slowly adding a heated water phase material to a heated oil phase material and agitating or homogenizing with a high-speed mixer. A variety of ingredients or combination of ingredients and active agents can be utilized to obtain a cosmetic formulation optimized for a particular utility or market segment and a reference source that lists standard cosmetic ingredients is the International Cosmetic Ingredient Dictionary and Handbook, published by The Cosmetic, Toiletry, and Fragrance Association, John A. Wenninger and G. N. McEwen, Jr., Editors, 7th Edition, 1997.

In one example embodiment, the antimicrobial composition may be made by initially preparing a precursor composition including a surfactant system with an HLB value of less than about 10, and an aqueous hydrophilic component. Addition to the precursor composition of an antimicrobial compound chosen from biguanides, (bis)biguanides, and mixtures and combinations thereof forms a stable microemulsion at room temperature with a disperse phase having droplets with a particle size of about 5 nm to about 400 nm. The microemulsion is stable for at least 6 months at room temperature, and the antimicrobial compound is present in the microemulsion in an amount sufficient to provide at least a 1.5-log microbial reduction on mammalian skin following 10 minute contact as measured according to ASTM E1874-09. To provide low flammability, the microemulsion includes less than about 10 wt % of lower monohydric alcohols, based on the total weight of the microemulsion.

Embodiments of the invention will now be illustrated with reference to the following non-limiting examples.

### EXAMPLES

### Example 1

This example compares the physical form and stability of oil/water/surfactant mixtures in the presence and absence of CHG. Isopropyl myristate (Jeen Chemical) was chosen as the oil phase, glycerol monocaprylate (Sasol Inc.) was chosen as the surfactant, and 20% CHG solution was obtained from Medichem Inc. As shown in Table 1 below, the following formulations were prepared:

TABLE 1

| | Without CHG | With CHG |
|---|---|---|
| Component | % w/w | % w/w |
| Isopropyl Myristate | 52.5 | 52.5 |
| Glyceryl Monocaprylate | 18.0 | 18.0 |
| Chlorohexidine Gluconate | 0 | 2.3 |
| Water | 29.5 | 27.2 |

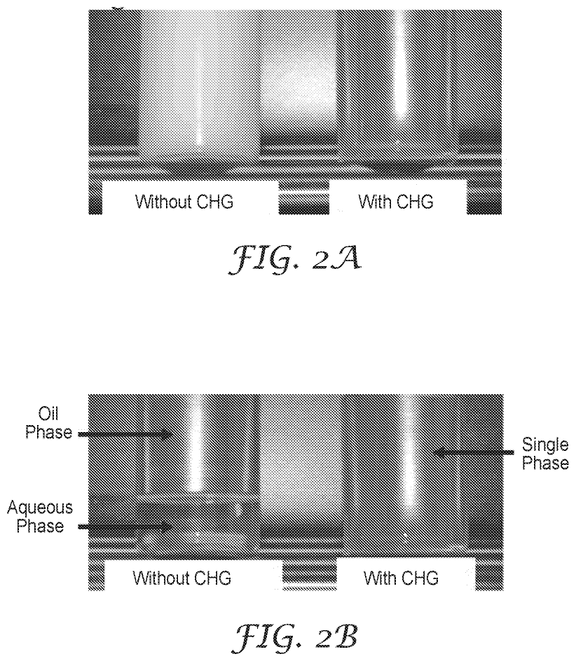

The formulation without CHG formed a milky white emulsion that separated into two phases on standing (see FIGS. 2A-2B). The formulation with CHG formed a clear, stable, isotropic microemulsion demonstrating the influence of CHG as both an antimicrobial compound and a cosurfactant.

### Example 2

This example demonstrates the importance of surfactant composition in the formation of a stable microemulsion. Compendial medium chain monoglycerides contain a mix of mono-, di-, and triglycerides of caprylic (C8) and capric (C10) acids. The mixture, which is predominantly monoglyceride, is commercially available from Abitec Corp. under the tradename CAPMUL. Four different grades of MCM were used to vary both the fatty acid profile as well as the fraction of monoglycerides. The nominal fatty acid composition and monoglyceride content of THE different grades is shown below.

### Capmul MCM

| | |
|---|---|
| Alpha Monocaprylocaprate | 48% min. |

### Capmul MCM NF

| | |
|---|---|
| Monoglycerides (%) | 49.5-60.5 (Target 55) |

Composition of Fatty Acids (wt. %)

| | |
|---|---|
| Caproic Acid | 1 max. |
| Caprylic Acid | 82-88 |
| Capric Acid | 12-18 |
| Lauric Acid and higher | 1 max. |

### Capmul MCM C8 EP

Composition of Fatty Acids (wt. %)

| | | |
|---|---|---|
| Caproic Acid | 1.0% | max. |
| Caprylic Acid | 90.0% | min. |
| Capric Acid | 10.0% | max. |
| Lauric Acid | 1.0% | max. |
| Myristic Acid | 0.5% | max. |

Content/Assay

| | |
|---|---|
| Monoacylglycerols | 45.0-75.0% |
| Diacylglycerols | 20.0-50.0% |
| Triacylglycerols | 10.0 |

### Capmul MCM C10

Composition of Fatty Acids (wt. %)

| Caproic Acid | 1.0 | max. |
|---|---|---|
| Caprylic Acid | 5.0 | max. |
| Capric Acid | 95.0 | min. |
| Lauric Acid | 3.0 | max. |
| Monoacylglycerols | 45.0-75.0% | |
| Diacylglycerols | 20.0-50.0% | |
| Triacylglycerols | 10.0% | max. |

The fraction of C8 monoglyceride in these emulsifiers follows the order MCM<MCM NF<MCM C8 EP˜MCM C10.

Isopropyl myristate was chosen as the oil phase due its dry, non-oily feel. Pseudoternary phase diagrams were then constructed using three components; 20% CHG (aqueous), isopropyl myristate, and medium chain monoglycerides.

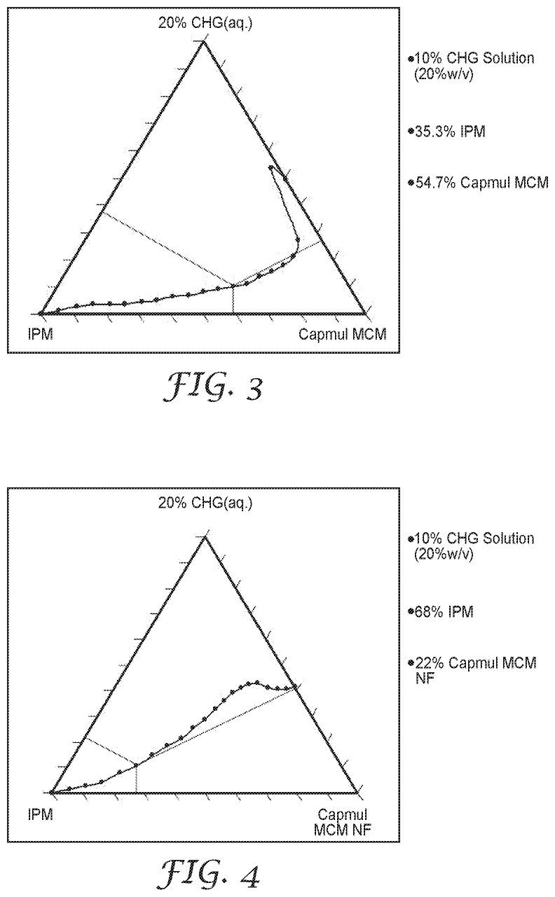

The phase diagram using MCM is shown in FIG. 3. The red tielines are for the 2% CHG (w/w) compositions; the relative lengths of the tielines denote the relative fractional contributions of the three components to the composition. The phase below the solid black line joining the individual datapoints in the diagram is a clear, isotropic microemulsion. Compositions above the line denote unstable macroemulsions. The vertices of the phase diagram represent 100% pure phase. The composition highlighted by the tielines is shown at the right of the phase diagram. FIG. 3 shows that the emulsifier phase is very large in the microemulsion and does not support the addition of much oil phase. An oil deficient microemulsion would not be able to efficiently plasticize drape adhesive and would have poor adhesion to skin under irrigation.

FIG. 4 shows the phase diagram when the MCM is replaced by the MCM NF. Note that an increase in the monoglyceride level has a drastic effect on the minimum amount of emulsifier required to form a microemulsion as the level has fallen from 54.7% to 22%.

FIG. 5 shows the phase diagram using MCM C8 EP as the emulsifier. This emulsifier has the highest caprylic acid content and highest fraction of monoglyceride.

Note that this combination affords the highest oil/emulsifier ratio. Use of the MCM C10 emulsifier did not result in microemulsion formation. The results point to a pure form of caprylic monoglyceride as the optimal emulsifier for this system.

### Example 3

Certain coemulsifiers are known to expand the microemulsion space in the phase diagram; these coemulsifiers are incapable of sustaining microemulsion formation with a primary emulsifier. Benzyl alcohol works particularly well in this function. The advantage of having an enhanced microemulsion space is that it affords greater latitude in microemulsion stability in the event of solvent (application on a wet skin site) and thermal excursions.

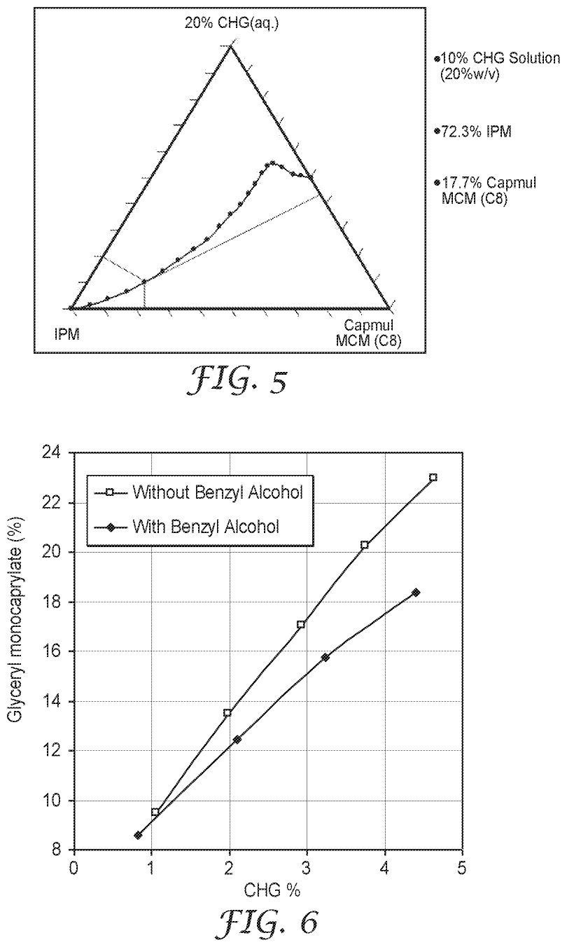

FIG. 6 shows the effect of the addition of 5% benzyl alcohol as a coemulsifier in the microemulsion system tested in FIG. 5 in Example 2 (MCM C8 EP was replaced with glyceryl monocaprylate from Sasol). The solid lines depict the threshold of the macroemulsion (below the line) to a microemulsion and represent the minimum amount of emulsifier needed for microemulsion formation. Note that the effect of benzyl alcohol increases with the level of CHG in the system.

### Example 4

Pigskin was used as a proxy for human skin to gauge the adhesive performance of Ioban incise drape over the prep under simulated irrigation conditions. The test method described in J. Bone Joint Surg. Am. 2012 Jul. 3; 94(13): 1187-92, “Comparison of two preoperative skin antiseptic preparations and resultant surgical incise drape adhesion to skin in healthy volunteers” was followed with the following exceptions.

Briefly, freshly euthanized pigs were clipped and shaved prior to prepping the skin with either ChloraPrep or the microemulsion prep formulation shown in Table 2 below:

TABLE 2

| | 2% CHG(w/v) |
|---|---|
| Component | % w/w |
| 1,2 Decanediol | 1.0 |
| Isopropyl Myristate | 52.5 |
| Benzyl Alcohol | 5.0 |
| Glyceryl Monocaprylate | 18.0 |
| Chlorohexidine Gluconate | 2.3 |
| Water | 21.2 |

Decanediol was added to the formulation for preservative action.

Each prepped area was allowed to dry for about 5 minutes and not more than 6 minutes. Strips cut 1.3 cm by 7.6 cm (0.5 in by 3 in) were applied in duplicate over the prepped area so that the long axis of the drape strip was orientated perpendicular to the pig's spine. To assure even application of the drape samples to the skin, a 2 kg (4.5 lb) roller was rolled over the drape samples once back and forth, using no additional pressure, immediately after the drape samples have been placed onto the test site. After the drape samples had been pressed in place with the roller, they were allowed to build adhesion for up to 5 minutes+/−30 seconds before any saline challenges were applied.

A 10 cm by 10 cm (4 in by 4 in) gauze that had been soaked in a 0.9% saline solution was placed over the drape sample immediately after the specified adhesion build time. Extra saline was added to the gauze at 10 minutes+/−2 minutes intervals during the challenge period to keep it saturated. The gauze was removed after 30 minutes+/−30 seconds. Immediately after removing the gauze from each sample, the drape sample was mechanically removed using a peel tester. The pull rate was 30.5 cm per minute (1 inches/min) at an angle of approximately 90 degrees to the skin. Data acquisition software was used to record the peel adhesion force. The results are shown in FIG. 7.

The skin adhesion of the Ioban incise drape to skin prepped with the non-flammable prep shows an improvement over ChloraPrep due to its amphiphilic nature and its ability to plasticize the drape adhesive.

### Example 5

### Antimicrobial Efficacy on Pig Skin

The pig skin used in 3M laboratories was obtained from the University of Minnesota Meat Lab. The pigs were rinsed with cold water to remove any gross contamination prior to removal of the belly skin. The skin was removed and immediately transported to 3M laboratories, where it was stretched and pinned to a large board. Any remaining gross contamination was gently removed with a damp cloth and the skin was dehaired using large animal clippers. A grid was taped down onto the skin to designate equally sized (2.5 in.×5 in.) testing sites. Prep formulations (6 ml) were applied to the skin by scrubbing for 30 seconds with a foam applicator. CHLOROPREP, a chlorhexidine gluconate and isopropyl alcohol preoperative skin preparation available from Becton Dickinson & Co., Franklin Lakes, N.J., with Tint 3 ml Applicator was used as a positive control, and was applied per manufacturer's instructions for a dry surgical site. Samples were collected from each test site after 10 minutes, using the cup scrub method as described in ASTM E1874-09.

The non-flammable skin prep described in Example 4 was evaluated for antimicrobial activity on porcine skin, using three different foam applicators. The applicator used foams that were precompressed to different ratios; applicator 1 had 2× compressed foam, applicator 2 had 6× and applicator 3 had 9× compressed foam. The compression is a post synthetic modification that changes the foam density. Each sample was tested in triplicate. The average baseline value across the pig skin was approximately 3.1 log₁₀ cfu/cm². Results are reported as log₁₀ reduction/cm².

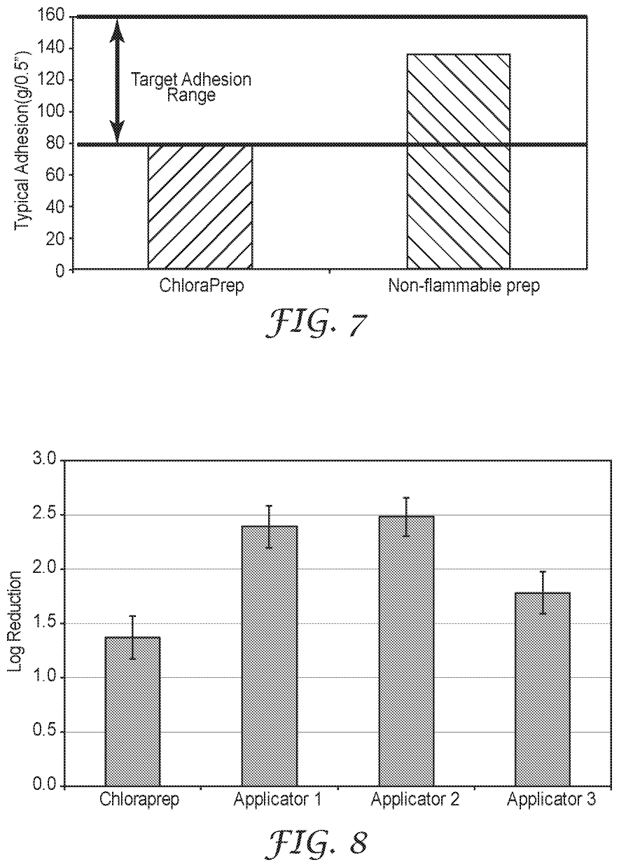

The results, which are plotted in FIG. 8, show that the non-flammable skin prep, when applied with applicators #1 and #2, reduce the microbial counts approximately 10-fold more than the CHLOROPREP control. Application with applicator #3 showed superior results to the CHLOROPREP control with regard to reducing microbial counts, however this type of foam applicator did not work as well as applicators #1 and #2.

### Example 6

This example demonstrates the non-flammability of the microemulsion formulations of the present disclosure. The non-flammable skin prep described in Example 4 was analyzed for Closed Cup Flash Point using ASTM D-3278-96 e-1 “Flash Point of Liquids by Small Scale Closed-Cup Apparatus.”

Results are shown in Table 3 below:

TABLE 3

| | | Comments/Observations |
|---|---|---|
| | | Test Method A (flash/no flash at a target temp.) |
| Sample | | Test Method B (used only when there is an |
| Identification | Flash @ | actual or finite flash point) |
| Micro- | No Flash | Test Method A. |
| emulsion | @ 70, | No Flash Point Identified. |
| prep with | 100, | In addition to testing at these specific |
| 2% CHG | 140 & | temperatures, the sample was ramped between |
| | 200° F. | them at a rate of 5° F./min. and was tested |
| | | every two degrees with no flash detected. |
| | | Testing was conducted at 718.15 mm Hg |
| | | barometric pressure. |
| | | The sample was a clear colorless liquid. |

### Example 7

The examples in Table 4 below illustrate various antimicrobial compositions using PHMB as the antimicrobial compound, alkanediols and blends with monoglycerides as amphiphilic surfactants, and a lipophilic component including higher alcohols.

TABLE 4

| Component | % w/w | % w/w | % w/w | % w/w | % w/w |
|---|---|---|---|---|---|
| Isopropyl Myristate | 40 | 40 | 40 | 52.5 | 40 |
| 1,2-Pentanediol | 10 | 10 | 5 | | |
| Glycerol monocaprylate | 16 | 18 | 15 | | 15 |
| 1,2-octanediol | 5 | 6 | | 17.5 | |
| Benzyl alcohol | 5 | 5 | 5 | | 5 |
| 1,2-decanediol | 1 | 1 | 1 | | 1 |
| Water | 13 | | | 20 | |
| 20% PHMB solution | 10 | 20 | | | |
| 20% CHG solution | | | 10 | 10 | 10 |
| Octyldodecanol | | | 9 | | |
| Ethylhexyl Isononanoate | | | 15 | | 24 |
| 1,2 hexanediol | | | | | 5 |

The microemulsions were formulated at room temperature by combining all the components except the antimicrobial. When this was complete, an unstable emulsions was formed, but addition of the antimicrobial transformed the unstable emulsion to a stable optically clear/translucent microemulsion.

Table 5 below contains a list of chemicals used for the examples 8-12 that follow.

TABLE 5

| Trade Name | Chemical Name | Supplier |
|---|---|---|
| Capmul 708-G | Glyceryl Caprylate | Abitec Corp. |
| Capmul PG-12 | Propylene Glycol Monolaurate | Abitec Corp. |
| Capmul MCM | Mono/diglycerides of caprylic/capric | Abitec Corp. |
| NF | acid | |
| Symdiol 68 | 1,2-Hexanediol (and) Caprylyl Glycol | Symrise |
| Sensiva SC50 | Ethylhexylglycerin | Schülke Inc. |
| CHG | chlorhexidine gluconate | Medichem |
| Kolliphor | Peg 15 hydroxystearate | Sigma Life |
| HS-15 | | Sciences |
| Gransolve DMI | Dimethyl Isosorbide | Gransolve |
| Tween 20 | Polysorbate 20 | Nikko |
| | | Chemicals Co. |
| IPM | Isopropyl Myristate | Inolex |
| Benzyl Alcohol | Benzyl Alcohol | |
| Sensiva SC-10 | Ethylhexyl glycerin and Caprylyl | Schülke Inc. |
| | glycol | |
| Propylene glycol | Propylene glycol | |
| Gluconolactone | | |
| Water | | |

### Example 8

Formulations 1 through 8 were prepared as described in Table 6 below.

TABLE 6

| | Formulas | #1 | #2 | #3 | #4 | #5 | #6 | #7 | #8 |
|---|---|---|---|---|---|---|---|---|---|
| Step 1 | Capmul 708 g | 20 | 20 | — | — | — | — | 20 | 20 |
| | Symdiol 68 | — | — | 20 | — | — | — | — | — |
| | Sensiva SC50 | — | — | — | 20 | — | — | — | — |
| | Capmul PG-12 | — | — | — | — | 20 | — | — | — |
| | Capmul MCM NF | — | — | — | — | — | 20 | — | — |
| Step 2 | Isopropyl Myristate | 30 | — | — | — | — | — | — | — |
| | Tween 20 | — | 12.5 | — | 5 | — | — | — | — |
| | Kolliphor HS15 | — | — | — | — | 20 | — | — | — |
| | Dimethyl Isosorbide | — | — | — | — | — | 10 | 10 | 10 |
| Step 3 | Benzyl Alcohol | — | 10 | — | — | 5 | — | — | 4 |
| Step 4 | Water | 40 | 47.5 | 70 | 65 | 45 | 60 | 60 | 56 |
| Step 5 | 20% CHG | 10 | 10 | 10 | 10 | 10 | 10 | 10 | 10 |
| | Appearance | clear | translucent | clear | clear | Slight | white | clear | translucent |
| | | | | | | haze | | | |

Many of the formulations in Table 6 formed clear or translucent microemulsions, in the absence of benzyl alcohol.

### Example 9

Placebo formulations (without CHG) were prepared as per Table 7 below.

TABLE 7

| | #1 | #2 | #3 | #4 | #5 | #6 | #7 | #8 |
|---|---|---|---|---|---|---|---|---|
| CHG (%) | 0 | 0 | 0 | 0 | 0 | 0 | 0 | 0 |
| Capmul 708-G (%) | 20 | 20 | | | | | 20 | 20 |
| Capmul PG-12 (%) | | | | | 20 | | | |
| Capmul MCM NF (%) | | | | | | 20 | | |
| Symdiol 68 (%) | | | 20 | | | | | |
| Sensiva SC50 (%) | | | | 20 | | | | |
| Kolliphor HS-15 (%) | | | | | 20 | | | |
| Dimethyl Isosorbide (%) | | | | | | 10 | 10 | 10 |
| Tween 20 (%) | | 12.5 | | 5 | | | | |
| Isopropyl Myristate (%) | 30 | | | | | | | |
| Benzyl Alcohol (%) | | 10 | | | 5 | | | 4 |
| Water (%) | 50 | 57.5 | 80 | 75 | 55 | 70 | 70 | 66 |
| Results | | | | | | | | |
| Phase | 2 | 2 | 2 | 2 | 2 | 2 | 2 | 2 |
| Fluidity | fluid | fluid | fluid | fluid | viscous | fluid | fluid | fluid |
| Clear Phases | 1 | | | 2 | | 1 | 1 | |
| Translucent Phases | | | | | | 1 | | 1 |
| Cloudy Phases | 1 | 2 | 2 | | 1 | | 1 | 1 |
| White Phases | | | | | 1 | | | |

None of the formulations in Table 7 above formed 1 clear, homogenous, stable phase in the absence of CHG.

### Example 10

Formulations were prepared with high levels of fatty acid monoesters and diols as set forth in Table 8 below.

TABLE 8

| | 1 | 2 | 3 | 4 | 5 |
|---|---|---|---|---|---|
| CHG (%) | 2 | 2 | 2 | 2 | 2 |
| Capmul 708-G (%) | 90 | | | | |
| Capmul PG-12 (%) | | 90 | | | |
| Capmul MCM NF (%) | | | 90 | | |
| Symdiol 68 (%) | | | | 90 | |
| Sensiva SC50 (%) | | | | | 90 |
| Water (%) | 8 | 8 | 8 | 8 | 8 |
| Results | | | | | |
| Phase | 1 | 2 | 1 | 1 | 1 |
| Fluidity | Fluid | Fluid | Fluid | Fluid | Fluid |
| Clear Phases | 1 | | 1 | 1 | 1 |
| Good Translucent | | 1 | | | |
| Phases | | | | | |
| Cloudy Phases | | 1 | | | |
| White Phases | | | | | |

Most of the solutions in Table 8 above formed micro-emulsions. Although the water content was reduced significantly in this experiment, stable micro-emulsions were formed.

### Example 11

Formulations of CHG were prepared with synergists and solubilizers like IPM, propylene glycol or DMI, as shown in Table 9 below.

TABLE 9

| | 1(g) | 2(g) | 3(g) | 4(g) |
|---|---|---|---|---|
| Chlorhexidine (20% w/v) | 10.65 | 10.65 | 10.65 | 10.65 |
| Capmul 708G | 10 | 2 | 8 | 10 |
| Isopropyl Myristate | 10 | 0 | 0 | 5 |
| Propylene Glycol | 0 | 10 | 10 | 0 |
| Dimethyl Isosorbide | 0 | 0 | 0 | 5 |
| Phenoxyethanol | 1 | 1 | 1 | 1 |
| Sensiva SC-10 | 0 | 1 | 1 | 1 |
| Gluconolactone | 0.2 | 0.2 | 0.2 | 0.2 |
| Water | 68.15 | 75.15 | 69.15 | 67.15 |
| Appearance | clear | clear | clear | clear |

None of the formulations of Table 9 formed stable solutions in the absence of CHG.

### Example 12

The octenidine formulations of Table 10 below were prepared with synergists in wt %.

TABLE 10

| | 1 | 2 | 3 | 4 | 5 |
|---|---|---|---|---|---|
| Octenidine | 0.3 | 0 | 0.3 | 0 | 0.3 |
| Capmul 708G | 10 | 10 | 2 | 2 | |
| Isopropyl Myristate | 10 | 10 | 0 | 0 | |
| Propylene Glycol | 0 | 0 | 10 | 10 | 20 |
| Benzyl alcohol | 5 | 5 | 0 | 0 | |
| Tween 20 | 10 | 10 | 1.2 | 1.2 | 1.5 |
| SC-10 | | | | | 5 |
| Appearance | Translucent | Cloudy | Clear | Translucent | Clear |

The clarity of the solution improved upon the addition of octenidine.

### Example 13

Visualization of the applied prep on the skin was enhanced by the addition of tints to the formulations. The formulations of Table 11 below employed two distinct tints that were soluble in either the oil phase (reddish yellow amber) or the water phase (green) of the formulations. The compositions of the tinted formulations are shown below in Table 11:

TABLE 11

| | 2% CHG | 3.5% CHG | 2% CHG | 4% CHG |
|---|---|---|---|---|
| Component | (% w/w) | (% w/w) | (% w/w) | (% w/w) |
| 1,2 Decanediol | 1.00 | 1.00 | 1.00 | 1.00 |
| Isopropyl Myristate | 52.50 | 55.08 | 52.30 | 52.10 |
| Benzyl Alcohol | | 5.00 | 5.00 | 5.00 |
| Glyceryl | 18.00 | 18.00 | 18.00 | 18.00 |
| Monocaprylate | | | | |
| Chlorhexidine | 2.18 | 3.74 | 2.18 | 4.36 |
| Gluconate* | | | | |
| Water | 25.40 | 16.26 | 21.32 | 18.94 |
| Sodium Gluconate | | | | 0.50 |
| FD&C Blue #1 | | | | 0.015 |
| FD&C Yellow #5 | | | | 0.085 |
| Beta Carotene** | 0.05 | 0.05 | 0.05 | |
| Apo-carotenal*** | 0.15 | 0.15 | 0.15 | |

*Chlorhexidine Gluconate was in a 20% (w/v) aqueous solution.
**Beta Carotene was in a 30% sunflower oil solution.
***The Apo-carotenal was in a 20% corn oil solution.

### Example 14

Microemulsions were prepared with dyes for increased patient compliance. The base compositions are shown in Table 12 below:

TABLE 12

| Component | Composition 1 (wt %) | Composition 2 (wt %) |
|---|---|---|
| CHG | 1-2 | 2 |
| Capmul 708G | 10 | 2 |
| DMI | 10 | |
| Benzyl Alcohol | 5 | |
| Isopropyl Alcohol | 4 | |
| Glucano-Lactone | 0.2 | 0.2 |
| Propylene Glycol | | 10 |
| Phenoxyethanol | | 1 |
| Sensiva SC-10 | | 1 |

Green 5 (0.01%) and Ultraphor (0.05%) were added to Composition 1 from Table 12, and formed a clear microemulsion with high intensity fluorescence when detected with a black light. When the same dye mixture was added to a comparative composition including 2% CHG and 10% propylene glycol, the comparative composition had 2 phases with a low intensity fluorescence signal.

0.1% red 28 was added to Composition 2 from Table 12, and resulted in an orange clear to translucent microemulsion.

Ultraphor at 0.1% was added to Composition 1 from Table 12, and resulted in a single phase clear microemulsion that was blue in color when irradiated with a black light.

Tinopal at 0.1% was added to Composition 1 from Table 12, and resulted in a composition that was blue in color when irradiated with a black light, but did not provide a clear microemulsion.

### EMBODIMENTS

A. A topical antimicrobial composition, comprising: a lipophilic component comprising a surfactant system with an HLB value of less than about 10; an amphiphilic component comprising an antimicrobial compound; an aqueous hydrophilic component; and a fluorescent material; wherein the composition comprises less than about 10 wt % of lower monohydric alcohols, based on the total weight of the composition, and wherein the composition is in the form of a microemulsion at room temperature with a disperse phase having droplets with a particle size of about 5 nm to about 400 nm.
B. The topical antimicrobial composition of Embodiment A, wherein the fluorescent material comprises a fluorescent dye with an absorption peak in the ultraviolet and violet portion of the electromagnetic spectrum (340-370 nm) with a corresponding emission peak in the visible portion of the spectrum (400-750 nm).
C. The topical antimicrobial composition of Embodiment A, wherein the fluorescent dye has an emission peak in the blue region of the visible spectrum (420-470 nm), and the emission peak is observable with an ultraviolet (UV) lamp.
D. The topical antimicrobial composition of Embodiment A, wherein the fluorescent material comprises a fluorescent dye with an emission peak in the near infrared (IR) region of the spectrum (780 nm to 2500 nm), and the emission peak is observable with an IR camera.
E. The topical antimicrobial composition of any of Embodiments A to D, wherein the fluorescent material comprises a fluorescent dye chosen from stilbene compounds, coumarin derivatives, diaryldipyrazoline derivatives, naphthalimide derivatives, benzoxazole derivatives, pyrazoline derivatives, cationic benzimidazole derivatives, hexasodium-2,2′-[vinylenebis[3-sulfonato-4,1-phenylene)imino[6-(diethylamino)-1,3,5-triazine-4,2-diyl]imino]]bis(benzene-1,4-disulphonate), and mixtures and combinations thereof.
F. The topical antimicrobial composition of any of Embodiments A to E, wherein the fluorescent material comprises a fluorescent glass.
G. The topical antimicrobial composition of any of Embodiments A to F, wherein the fluorescent material comprises a fluorescent dye present at about 0.001 wt % to about 10 wt %, based on the total weight of the composition.
H. The topical antimicrobial composition of any of Embodiments A to G, wherein the composition further comprises a visible dye with an absorption and transmission peak in the visible region of the electromagnetic spectrum (400-700 nm).
I. The topical antimicrobial composition of any of Embodiments A to H, wherein the fluorescent material comprises a fluorescent dye that is a visible dye.
J. The topical antimicrobial composition of any of Embodiments A to I, wherein the composition further comprises a colorant chosen from a pigment, a lake, a polymeric colorant, and mixtures and combinations thereof.
K. The topical antimicrobial composition of Embodiment J, wherein the colorant is a pigment.
L. The topical antimicrobial composition of any of Embodiment J, wherein the colorant is present in the composition at about 0.10% wt % to about 30% wt %, based on the total weight of the composition.
M. The topical antimicrobial composition of any of Embodiments A to L, wherein the microemulsion has a disperse phase with droplets having a particle size of about 5 nm to about 200 nm.
N. The topical antimicrobial composition of any of Embodiments A to L, wherein the microemulsion has a disperse phase with droplets having a particle size of about 5 nm to about 100 nm.
O. The topical antimicrobial composition of any of Embodiments A to N, wherein the microemulsion is a liquid at room temperature.
P. The topical antimicrobial composition of any of Embodiments A to N, wherein the microemulsion is a gel at room temperature.
Q. The topical antimicrobial composition of any of Embodiments A to P, wherein the composition comprises less than about 5% of lower monohydric alcohols.
R. The topical antimicrobial composition of any of Embodiments A to P, wherein the composition is substantially free of lower monohydric alcohols.
S. The topical antimicrobial composition of any of Embodiments A to R, wherein the microemulsion is translucent to visible light at room temperature.
T. The topical antimicrobial composition of any of Embodiments A to R, wherein the microemulsion is clear to visible light at room temperature.
U. The topical antimicrobial composition of any of Embodiments A to T, wherein the microemulsion is stable for at least 6 months at room temperature.
V. The topical antimicrobial composition of any of Embodiments A to T, wherein the microemulsion is stable for at least 2 years at room temperature.
W. The topical antimicrobial composition of any of Embodiments A to V, wherein the composition has no closed cup flash point at temperatures of 70° F. to 200° F. as measured according to ASTM D-3278-96 e-1.
X. The topical antimicrobial composition of any of Embodiments A to W, wherein the composition provides at least a 1.5-log microbial reduction on mammalian skin following 10 minute contact as measured according to ASTM E1874-09.
Y. The topical antimicrobial composition of any of Embodiments A to W, wherein the composition provides at least a 2-log microbial reduction on mammalian skin following 10 minute contact as measured according to ASTM E1874-09.
Z. The topical antimicrobial composition of any of Embodiments A to Y, wherein the composition adheres a surgical drape to mammalian skin at greater than about 80 grams per 0.5 inches as measured at a pull rate of 1 inch per minute at an angle of about 900 to the skin according to a modified test procedure from J. Bone, Joint Surg. Am. 2012 Jul. 3; 94(13): 1187-92.
AA. The topical antimicrobial composition of any of Embodiments A to Z, wherein the aqueous hydrophilic component is present in an amount of about 5 wt % to about 98 wt %, based on the total weight of the composition.
BB. The topical antimicrobial composition of any of Embodiments A to Z, wherein the aqueous hydrophilic component is present in an amount of about 10 wt % to about 90 wt %, based on the total weight of the composition.
CC. The topical antimicrobial composition of any of Embodiments A to Z, wherein the aqueous hydrophilic component comprises at least about 80 wt % water, based on the total weight of the aqueous hydrophilic component.
DD. The topical antimicrobial composition of any of Embodiments A to Z, wherein the aqueous hydrophilic component comprises at least about 90 wt % water, based on the total weight of the aqueous hydrophilic component.
EE. The topical antimicrobial composition of any of Embodiments A to Z, wherein the aqueous hydrophilic component consists of water.
FF. The topical antimicrobial composition of any of Embodiments A to EE, wherein the aqueous hydrophilic component comprises glycerol, propylene glycol, polyethylene glycol, pentylene glycol, and mixtures and combinations thereof.
GG. The antimicrobial composition of any of Embodiments A to FF, wherein the lipophilic component is present in the composition at about 0.5 wt % to about 50 wt %, based on the total weight of the composition.
HH. The antimicrobial composition of any of Embodiments A to FF, wherein the lipophilic component is present in the composition at about 5 wt % to about 35 wt %, based on the total weight of the composition.
II. The antimicrobial composition of any of Embodiments A to FF, wherein the lipophilic component is present in the composition at about 1 wt % to about 30 wt %, based on the total weight of the composition.
JJ. The topical antimicrobial composition of any of Embodiments A to II, wherein the lipophilic component is chosen from esters, ethers, amides, glycols, monoalkyl and monoalkylene alcohols with greater than 7 carbons atoms and less than 18 carbon atoms, liquid paraffins, liquid waxes, and mixtures and combinations thereof.
KK. The topical antimicrobial composition of Embodiment JJ, wherein the lipophilic component is chosen from an ester, an ether, a glycol, and mixtures and combinations thereof.
LL. The topical antimicrobial composition of Embodiment KK, wherein the ester is chosen from isopropyl myristate, isopropyl palmitate, dibutyl adipate, diisobutyl adipate, fatty acid esters of glycerol, fatty acid esters of propylene glycol, and mixtures and combinations thereof.
MM. The topical antimicrobial composition of Embodiment LL, wherein the fatty ester of glycerol is chosen from glyceryl mono, di, and tri caprylate, and mixtures and combinations thereof.
NN. The topical antimicrobial composition of any of Embodiments KK to MM, wherein the ether is ethylhexyl glycerin.
OO. The topical antimicrobial composition of any of Embodiments KK to NN, wherein the glycol is chosen from 1, 2 octane diol, 1,2 decane diol, and mixtures and combinations thereof.
PP. The topical antimicrobial composition of any of Embodiments A to OO, wherein the amphiphilic component is present in the composition at about 0.05 wt % to about 40 wt %, based on the total weight of the composition.
QQ. The topical antimicrobial composition of any of Embodiments A to OO, wherein the amphiphilic component is present in the composition at about 1 wt % to about 30 wt %, based on the total weight of the composition.
RR. The topical antimicrobial composition of any of Embodiments A to QQ, wherein the surfactant system comprises an ester of glycerol with a fatty acid.
SS. The topical antimicrobial composition of claim Embodiment RR, wherein the fatty acid is chosen from oleic, linoleic, linolenic, caproic, caprylic, capric, lauric, and mixtures and combinations thereof.
TT. The topical antimicrobial composition of any of Embodiments RR to SS, wherein the surfactant system is chosen from caprylic, capric, and mixtures and combinations thereof.
UU. The topical antimicrobial composition of Embodiment TT, wherein the fatty acid comprises a mixture of monoglycerides, diglycerides, and triglycerides of caprylic acid and capric acid.
VV. The topical antimicrobial composition of Embodiment TT, wherein the fatty acid comprises: at least 80% by weight of a mixture of monoglycerides of caprylic acid; and up to about 20% by weight of a mixture of monoglycerides of capric acid.
WW. The topical antimicrobial composition of Embodiment TT, wherein the fatty acid comprises: at least 90% by weight of a mixture of monoacylglycerols, diacylglycerols, and triacylglycerols of caprylic acid; and about 10% by weight of a mixture of monoacylglycerols, diacylglycerols, and triacylglycerols of capric acid.
XX. The topical antimicrobial composition of Embodiment TT, wherein the fatty acid comprises: at least about 95% by weight of a mixture of monoacylglycerols, diacylglycerols, and triacylglycerols of capric acid; and about 5% by weight of a mixture of monoacylglycerols, diacylglycerols, and triacylglycerols of caprylic acid.
YY. The topical antimicrobial composition of any of Embodiments A to XX, wherein the antimicrobial compound is chosen from octenidine, biguanides, (bis)biguanides, and mixtures and combinations thereof.
ZZ. The topical antimicrobial composition of Embodiment YY, wherein the antimicrobial compound is chosen from octenidine, PHMB, CHG, and combinations thereof.
AAA. The topical antimicrobial composition of Embodiment YY, wherein the antimicrobial compound is CHG.
BBB. The topical antimicrobial composition of any of Embodiments A to AAA, wherein the antimicrobial compound is present in the composition at about 0.05% by weight to about 10% by weight, based on the total weight of the composition.
CCC. The topical antimicrobial composition of any of Embodiments A to AAA, wherein the antimicrobial compound is present in the composition at about 0.05% by weight to about 5% by weight, based on the total weight of the composition.
DDD. The topical antimicrobial composition of any of Embodiments A to CCC, wherein the composition further comprises an alcohol chosen from benzyl alcohol, phenoxy ethanol, isopropyl alcohol, ethanol and combinations thereof.
EEE. The topical antimicrobial composition of Embodiment DDD, wherein the alcohol is present in the composition at about 1 wt % to about 10 wt %, based on the total weight of the composition.
FFF. The topical antimicrobial composition of any of Embodiments A to EEE, further comprising a preservative.
GGG. The topical antimicrobial composition of any of Embodiments A to FFF, further comprising a humectant.
HHH. A topical mammalian tissue antiseptic composition, the composition comprising: about 2 wt % to about 50 wt %, based on the total weight of the composition, of an ester chosen from isopropyl myristate, isopropyl palmitate, dibutyl adipate, diisobutyl adipate, monoalkyl glycols; glycerol alkyl ethers; monoacyl glycerols, and mixtures and combinations thereof, about 0.5 wt % to about 10 wt %, based on the total weight of the composition, of an antimicrobial compound chosen from biguanides, (bis)biguanides, octenidine, and mixtures and combinations thereof, about 5 wt % to about 95 wt %, based on the total weight of the composition, of an aqueous hydrophilic component comprising at least 80 wt % water, based on the total weight of the aqueous hydrophilic component; and a fluorescent material; wherein the composition comprises less than about 5 wt % of lower monohydric alcohols, based on the total weight of the composition, wherein the composition is in the form of an oil in water microemulsion at room temperature, and wherein the microemulsion has a disperse phase having droplets with a particle size of about 5 nm to about 400 nm.
III. The topical mammalian tissue antiseptic composition of Embodiment HHH, wherein the composition further comprises at least 5% propylene glycol.
JJJ. The topical mammalian tissue antiseptic composition of any of Embodiments HHH to III, wherein the composition comprises monoalkyl glycols.
KKK. The topical mammalian tissue antiseptic composition of any of Embodiments HHH to JJJ, wherein the antimicrobial compound is chosen from PHMB, CHG, and combinations thereof.
LLL. The topical mammalian tissue antiseptic composition of Embodiment KKK, wherein the antimicrobial compound is CHG.
MMM. The topical mammalian tissue antiseptic composition of any of Embodiments HHH to LLL, wherein the antimicrobial compound is present in the composition at about 0.5% wt % to about 5% wt %, based on the total weight of the composition.
NNN. The topical mammalian tissue antiseptic composition of any of Embodiments HHH to LLL, wherein the aqueous hydrophilic component comprises 100 wt % water.
OOO. A method of disinfecting mammalian skin, the method comprising: applying to the mammalian skin a topical antiseptic composition comprising: a lipophilic component comprising a surfactant system with an HLB value of less than about 10; an amphiphilic component comprising an antimicrobial compound; an aqueous hydrophilic component; and a fluorescent dye; wherein the composition comprises less than about 10 wt % of lower monohydric alcohols, based on the total weight of the composition, and wherein the composition is in the form of a microemulsion at room temperature with a disperse phase having droplets with a particle size of about 5 nm to about 400 nm.
PPP. The method of Embodiment OOO, wherein the fluorescent dye has an absorption peak in the ultraviolet and violet portion of the electromagnetic spectrum (340-370 nm) with a corresponding emission peak in the visible portion of the spectrum (400-750 nm).
QQQ. The method of any of Embodiment OOO, wherein the fluorescent dye has an emission peak in the blue region of the visible spectrum (420-470 nm), and the emission peak is observable with an ultraviolet (UV) lamp.
RRR. The method of Embodiment OOO, wherein the fluorescent dye has an emission peak in the near infrared (IR) region of the spectrum (780 nm to 2500 nm), and the emission peak is observable with an IR camera.
SSS. The method of any of Embodiments OOO to RRR, wherein the fluorescent dye is chosen from stilbene compounds, coumarin derivatives, diaryldipyrazoline derivatives, naphthalimide derivatives, benzoxazole derivatives, pyrazoline derivatives, cationic benzimidazole derivatives, hexasodium-2,2′-[vinylenebis[3-sulfonato-4,1-phenylene)imino[6-(diethylamino)-1,3,5-triazine-4,2-diyl]imino]]bis(benzene-1,4-disulphonate), and mixtures and combinations thereof.
TTT. The method of any of Embodiments OOO to SSS, wherein the fluorescent dye comprises a fluorescent glass.
UUU. The method of any of Embodiments OOO to TTT, wherein the fluorescent dye is present at about 0.001 wt % to about 10 wt %, based on the total weight of the composition.
VVV. The method of any of Embodiments OOO to UUU, wherein the composition further comprises a visible dye with an absorption and transmission peak in the visible region of the electromagnetic spectrum (400-700 nm).
WWW. The method of any of Embodiments OOO to VVV, wherein the fluorescent dye is a visible dye.
XXX. The method of any of Embodiments OOO to WWW, wherein the composition further comprises a colorant chosen from a pigment, a lake, a polymeric colorant, and mixtures and combinations thereof.
YYY. The method of Embodiment XXX, wherein the colorant is a pigment.
ZZZ. The method of any of Embodiments XXX to YYY, wherein the colorant is present in the composition at about 0.10% wt % to about 30% wt %, based on the total weight of the composition.
AAAA. The method of any of Embodiments OOO to ZZZ, wherein the topical antiseptic composition is applied to the skin with at least one of a non-woven mitt, a glove, a sponge, and a cloth.
BBBB. The method of Embodiment AAAA, wherein the topical antiseptic composition is impregnated in the mitt.
CCCC. The method of any of Embodiments AAAA to BBBB, wherein the topical antiseptic composition is encapsulated in a particle within a surface of the mitt.
DDDD. The method of any of Embodiments OOO to CCCC, further comprising applying a surgical incise drape on the topical antiseptic composition.
EEEE. The method of any of Embodiments OOO to DDDD, wherein applying to the mammalian skin comprises applying a surgical incise drape to the skin, wherein a surface of the surgical incise drape contacting the skin is impregnated with the topical antiseptic composition.
FFFF. The method of any of Embodiments OOO to EEEE, wherein the topical antiseptic composition has no closed cup flash point at temperatures of 70° F. to 200° F. as measured according to ASTM D-3278-96 e-1.
GGGG. The method of any of Embodiments OOO to FFFF, wherein the topical antiseptic composition provides at least a 1.5-log microbial reduction following 10 minute contact as measured according to ASTM E1874-09.
HHHH. The method of any of Embodiments OOO to FFFF, wherein the topical antiseptic composition provides at least a 2-log microbial reduction following 10 minute contact as measured according to ASTM E1874-09.
IIII. The method of any of Embodiments DDDD to HHHH, wherein the topical antiseptic composition adheres to the surgical incise drape at greater than about 80 grams per 0.5 inches as measured at a pull rate of 1 inch per minute at an angle of about 900 to the skin according to a modified test procedure from J. Bone, Joint Surg. Am. 2012 Jul. 3; 94(13): 1187-92.
JJJJ. A kit, comprising: a topical mammalian tissue antiseptic composition, comprising: a lipophilic component comprising a surfactant system with an HLB value of less than about 10; an amphiphilic component comprising an antimicrobial compound; an aqueous hydrophilic component; and a fluorescent dye; wherein the composition comprises less than about 10 wt % of lower monohydric alcohols, based on the total weight of the composition, and wherein the composition is in the form of a microemulsion at room temperature with a disperse phase having droplets with a particle size of about 5 nm to about 400 nm; and an applicator for applying the antiseptic composition to skin of a patient.
KKKK. The kit of Embodiment JJJJ, further comprising a surgical incise drape.
LLLL. The kit of any of Embodiments JJJJ to KKKK, further comprising a tray containing a container of the antiseptic composition, the applicator, and the surgical incise drape.
MMMM. The kit of any of Embodiments JJJJ to LLLL, wherein the kit further comprises instructions for applying the antiseptic composition.
NNNN. The kit of any of Embodiments JJJJ to MMMM, wherein the applicator is chosen from a mitt, a glove, a sponge, and a cloth.
OOOO. The kit of Embodiment NNNN, wherein the applicator is a non-woven mitt.

Various embodiments of the invention have been described. These and other embodiments are within the scope of the following claims.

1. A topical antimicrobial composition, comprising:
a lipophilic component comprising a surfactant system with an HLB value of less than about 10;
an amphiphilic component comprising an antimicrobial compound;
an aqueous hydrophilic component; and
a fluorescent material;
wherein the composition comprises less than about 10 wt % of lower monohydric alcohols, based on the total weight of the composition, and wherein the composition is in the form of a microemulsion at room temperature with a disperse phase having droplets with a particle size of about 5 nm to about 400 nm.

2. The topical antimicrobial composition of claim 1, wherein the fluorescent material comprises a fluorescent dye with an absorption peak in the ultraviolet and violet portion of the electromagnetic spectrum (340-370 nm) with a corresponding emission peak in the visible portion of the spectrum (400-750 nm).

3. The topical antimicrobial composition of claim 2, wherein the fluorescent dye has an emission peak in the blue region of the visible spectrum (420-470 nm), and the emission peak is observable with an ultraviolet (UV) lamp.

4. The topical antimicrobial composition of claim 1, wherein the fluorescent material comprises a fluorescent dye with an emission peak in the near infrared (IR) region of the spectrum (780 nm to 2500 nm), and the emission peak is observable with an IR camera.

5. The topical antimicrobial composition of claim 1, wherein the fluorescent material comprises a fluorescent dye chosen from stilbene compounds, coumarin derivatives, diaryldipyrazoline derivatives, naphthalimide derivatives, benzoxazole derivatives, pyrazoline derivatives, cationic benzimidazole derivatives, hexasodium-2,2′-[vinylenebis[3-sulfonato-4,1-phenylene)imino[6-(diethylamino)-1,3,5-triazine-4,2-diyl]imino]]bis(benzene-1,4-disulphonate), curcuminoids, and mixtures and combinations thereof.

6. The topical antimicrobial composition of claim 1, wherein the fluorescent material comprises a fluorescent dye present at about 0.001 wt % to about 10 wt %, based on the total weight of the composition.

7. The topical antimicrobial composition of claim 1, wherein the composition further comprises a visible dye with an absorption and transmission peak in the visible region of the electromagnetic spectrum (400-700 nm).

8. (canceled)

9. The topical antimicrobial composition of claim 1, wherein the composition comprises less than about 5% of lower monohydric alcohols.

10. The topical antimicrobial composition of claim 1, wherein the composition has no closed cup flash point at temperatures of 70° F. to 200° F. as measured according to ASTM D-3278-96 e-1.

11. The topical antimicrobial composition of claim 1, wherein the composition provides at least a 1.5-log microbial reduction on mammalian skin following 10 minute contact as measured according to ASTM E1874-09.

12. The topical antimicrobial composition of claim 1, wherein the aqueous hydrophilic component is present in an amount of about 5 wt % to about 98 wt %, based on the total weight of the composition.

13. The topical antimicrobial composition of claim 1, wherein the aqueous hydrophilic component comprises at least about 80 wt % water, based on the total weight of the aqueous hydrophilic component.

14. The antimicrobial composition of claim 1, wherein the lipophilic component is present in the composition at about 0.5 wt % to about 50 wt %, based on the total weight of the composition.

15. The topical antimicrobial composition of claim 1, wherein the lipophilic component is chosen from esters, ethers, amides, glycols, monoalkyl and monoalkylene alcohols with greater than 7 carbons atoms and less than 18 carbon atoms, liquid paraffins, liquid waxes, and mixtures and combinations thereof.

16. The topical antimicrobial composition of claim 1, wherein the amphiphilic component is present in the composition at about 0.05 wt % to about 40 wt %, based on the total weight of the composition.

17. The topical antimicrobial composition of claim 1, wherein the surfactant system comprises an ester of glycerol with a fatty acid.

18. The topical antimicrobial composition of claim 1, wherein the antimicrobial compound is chosen from octenidine, biguanides, (bis)biguanides, polymeric biguanides, quaternary ammonium salts, and mixtures and combinations thereof.

19. The topical antimicrobial composition of claim 1, wherein the antimicrobial compound is present in the composition at about 0.05% by weight to about 10% by weight, based on the total weight of the composition.

20. A method of disinfecting mammalian skin, the method comprising:
applying to the mammalian skin the topical antiseptic composition of claim 1.

21. A kit, comprising:
the topical mammalian tissue antiseptic composition of claim 1; and
an applicator for applying the antiseptic composition to skin of a patient.
